SEQUENCE LISTING (20 sequences)

SEQ ID NO: 1 - protein, 294 aa, Pseudomonas aeruginosa
MEVRNFNAGRELFLDELLAAPAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLRDKYPFEKDNNPVGNFATTVSDRSRPLNDKVNEKTTLLNDTSSRYNSAVEALNRFIQKYDSVLRDILSAI

SEQ ID NO: 2 - DNA, 882 bp, Pseudomonas aeruginosa
atggaagtcagaaactttaatgccggacgcgagctgttcctggacgaactcctggccgcgccggcggcgcctgccagtgccgagcaggaggaactgctggccttgttgcgcagcgagcggatcgtgctggcacacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatttgcgcgagttcctggtgtcggcctatttcagcctgcacggccgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaagcagggcatcaggatcgacgctggcggtatcgatctggtcgaccccacgctctatggctatgccgtcggcgatcccaggtggaaggacagccccgagtatgcgctgctgagcaatctggataccttcagcggcaagctgtcgatcaaggattttctcagcggctcgccgaagcagagcggggaactcaagggccttagggataagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgcgcgacattctcagcgcgatc

SEQ ID NO: 3 - protein, 153 aa, Pseudomonas aeruginosa
APAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVE

SEQ ID NO: 4 - DNA, 458 bp, Pseudomonas aeruginosa
ctccggctccggaaccggttgccgacgtttgctccgactccgacaacgacggcgtttgcgacaacgtcgacaagtgcccggataccccggccaacgtcaccgttgacgccaacggctgcccggctgtcgccgaagtcgtacgcgtacagctggacgtgaagttcgacttcgacaagtccaaggtcaaagagaacagctacgctgacatcaagaacctggctgacttcatgaagcagtacccgtccacttccaccaccgttgaaggtcacaccgactccgtcggcaccgacgcttacaaccagaagctgtccgagcgtcgtgccaacgccgttcgtgacgtactggtcaacgagtacggtgtagaaggtggtcgcgtgaacgctgttggttacggcgagtcccgcccggttgccgacaacgccaccgctgaaggccgcgctatcaaccgtcgcgttgaa

SEQ ID NO: 5 - protein, 208 aa, Pseudomonas aeruginosa
DVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 6 - protein, 627 aa, Pseudomonas aeruginosa
GACGTCAGCTTCAGCACCCGCGGCACGCAGAACTGGACGGTGGAGCGGCTGCTCCAGGCGCACCGCCAACTGGAGGAGCGCGGCTATGTGTTCGTCGGCTACCACGGCACCTTCCTCGAAGCGGCGCAAAGCATCGTCTTCGGCGGGGTGCGCGCGCGCAGCCAGGACCTCGACGCGATCTGGCGCGGTTTCTATATCGCCGGCGATCCGGCGCTGGCCTACGGCTACGCCCAGGACCAGGAACCCGACGCACGCGGCCGGATCCGCAACGGTGCCCTGCTGCGGGTCTATGTGCCGCGCTCGAGCCTGCCGGGCTTCTACCGCACCAGCCTGACCCTGGCCGCGCCGGAGGCGGCGGGCGAGGTCGAACGGCTGATCGGCCATCCGCTGCCGCTGCGCCTGGACGCCATCACCGGCCCCGAGGAGGAAGGCGGGCGCCTGGAGACCATTCTCGGCTGGCCGCTGGCCGAGCGCACCGTGGTGATTCCCTCGGCGATCCCCACCGACCCGCGCAACGTCGGCGGCGACCTCGACCCGTCCAGCATCCCCGACAAGGAACAGGCGATCAGCGCCCTGCCGGACTACGCCAGCCAGCCCGGCAAACCGCCGCGCGAGGACCTGAAGTAA

SEQ ID NO: 7 - protein, 207 aa, Artificial Sequence
  mTox 578E deletion
DVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLTILGWPLAERTVVIPSAIPTDPRNVGGDLDPSSIPDKEQAISALPDYASQPGKPPREDLK

SEQ ID NO: 8 - DNA, 624 bp, Artificial Sequence
  mTox 578E deletion
gacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctgaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaa

SEQ ID NO: 9 - protein, 5 aa, Artificial Sequence
  GS linker 1
GGGGS

SEQ ID NO: 10 - protein, 6 aa, Artificial Sequence
  GS linker 2
GSGGSG

SEQ ID NO: 11 - protein, 665 aa, Artificial Sequence
  POmT
MEVRNFNAGRELFLDELLAAPAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLRDKYPFEKDNNPVGNFATTVSDRSRPLNDKVNEKTTLLNDTSSRYNSAVEALNRFIQKYDSVLRDILSAIGGGGSAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEGSGGSGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLTILGWPLAERTVVIPSAIPTDPRNVGGDLDPSSIPDKEQAISALPDYASQPGKPPREDLK

SEQ ID NO: 12 - DNA, 1998 bp, Artificial Sequence
  POmT
atggaagtcagaaactttaatgccggacgcgagctgttcctggacgaactcctggccgcgccggcggcgcctgccagtgccgagcaggaggaactgctggccttgttgcgcagcgagcggatcgtgctggcacacgccggccagccgctgagcgaggcgcaagtgctcaaggcgctcgcctggttgctcgcggccaatccgtccgcgcctccggggcagggcctcgaggtactccgcgaagtcctgcaggcacgtcggcagcccggtgcgcagtgggatttgcgcgagttcctggtgtcggcctatttcagcctgcacggccgtctcgacgaggatgtcatcggtgtctacaaggatgtcctgcagacccaggacggcaagcgcaaggcgctgctcgacgagctcaaggcgctgaccgcggagttgaaggtctacagcgtgatccagtcgcagatcaacgccgcgctgtcggccaagcagggcatcaggatcgacgctggcggtatcgatctggtcgaccccacgctctatggctatgccgtcggcgatcccaggtggaaggacagccccgagtatgcgctgctgagcaatctggataccttcagcggcaagctgtcgatcaaggattttctcagcggctcgccgaagcagagcggggaactcaagggccttagggataagtaccccttcgagaaggacaacaacccggtcggcaatttcgccaccacggtgagcgaccgctcgcgtccgctgaacgacaaggtcaacgagaagaccaccctgctcaacgacaccagctcccgctacaactcggcggtcgaggcgctcaaccgcttcatccagaaatacgacagcgtcctgcgcgacattctcagcgcgatcggcggtggcggatccgctccggctccggaaccggttgccgacgtttgctccgactccgacaacgacggcgtttgcgacaacgtcgacaagtgcccggataccccggccaacgtcaccgttgacgccaacggctgcccggctgtcgccgaagtcgtacgcgtacagctggacgtgaagttcgacttcgacaagtccaaggtcaaagagaacagctacgctgacatcaagaacctggctgacttcatgaagcagtacccgtccacttccaccaccgttgaaggtcacaccgactccgtcggcaccgacgcttacaaccagaagctgtccgagcgtcgtgccaacgccgttcgtgacgtactggtcaacgagtacggtgtagaaggtggtcgcgtgaacgctgttggttacggcgagtcccgcccggttgccgacaacgccaccgctgaaggccgcgctatcaaccgtcgcgttgaaggttcagggggttccggtgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctgaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgcgcgaggacctgaagtaa

SEQ ID NO: 13 - protein, 14 aa, Artificial Sequence
  Tag
MKHHHHHHHMHAKM

SEQ ID NO: 14 - protein, 294 aa, Pseudomonas aeruginosa
MEVRNLNAARELFLDELLAASAAPASAEQEELLALLRSERIVLAHAGQPLSEAQVLKALAWLLAANPSAPPGQGLEVLREVLQARRQPGAQWDLREFLVSAYFSLHGRLDEDVIGVYKDVLQTQDGKRKALLDELKALTAELKVYSVIQSQINAALSAKQGIRIDAGGIDLVDPTLYGYAVGDPRWKDSPEYALLSNLDTFSGKLSIKDFLSGSPKQSGELKGLSDEYPFEKDNNPVGNFATTVSDRSRPLNDKVNEKTTLLNDTSSRYNSAVEALNRFIQKYDSVLRDILSAI

SEQ ID NO: 15 - protein, 350 aa, Pseudomonas aeruginosa
MKLKNTLGVVIGSLVAASAMNAFAQGQNSVEIEAFGKRYFTDSVRNMKNADLYGGSIGYFLTDDVELALSYGEYHDVRGTYETGNKKVHGNLTSLDAIYHFGTPGVGLRPYVSAGLAHQNITNINSDSQGRQQMTMANIGAGLKYYFTENFFAKASLDGQYGLEKRDNGHQGEWMAGLGVGFNFGGSKAAPAPEPVADVCSDSDNDGVCDNVDKCPDTPANVTVDANGCPAVAEVVRVQLDVKFDFDKSKVKENSYADIKNLADFMKQYPSTSTTVEGHTDSVGTDAYNQKLSERRANAVRDVLVNEYGVEGGRVNAVGYGESRPVADNATAEGRAINRRVEAEVEAEAK

SEQ ID NO: 16 - protein, 638 aa, Pseudomonas aeruginosa
MHLIPHWIPLVASLGLLAGGSSASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 17 - protein, 5 aa, Artificial Sequence
  GS unit
GGGGS

SEQ ID NO: 18 - protein, 4 aa, Artificial Sequence
  GS unit
SAGG

SEQ ID NO: 19 - protein, 4 aa, Artificial Sequence
  GS unit
GGGS

SEQ ID NO: 20 - protein, 4 aa, Artificial Sequence
  GS unit
GGSG

(19) United States
(12) Patent Application Publication
(10) Pub. No.: US 2023/0241197 A1
(43) Pub. Date: Aug. 3, 2023

(54) PROTEIN MOLECULE USEFUL FOR ANTI-PSEUDOMONAS AERUGINOSA VACCINE

(71) Applicant: KYOTO PREFECTURAL PUBLIC UNIVERSITY CORPORATION, Kyoto (JP)

(72) Inventor: Teiji SAWA, Kyoto (JP)

(73) Assignee: KYOTO PREFECTURAL PUBLIC UNIVERSITY CORPORATION, Kyoto (JP)

(21) Appl. No.: 17/927,199

(22) PCT Filed: May 13, 2021
(86) PCT No.: PCT/JP2021/018141

§ 371 Date: Nov. 22, 2022

(30) Foreign Application Priority Data
May 27, 2020 (JP) 2020-092376

(51) Int. Cl. A61K 39/104; C07K 14/21; C07K 16/12; A61P 31/04

(52) U.S. Cl. CPC A61K 39/104; C07K 14/21; C07K 16/1214; A61P 31/04; C07K 2319/00

(57) ABSTRACT

The present invention provides a protein molecule effective for an anti-Pseudomonas aeruginosa vaccine. A protein molecule comprising PcrV antigen domain and at least one domain selected from the group consisting of OprF antigen domains and Exotoxin A antigen domains.

### TECHNICAL FIELD

The present invention relates to a protein molecule that is effective for an anti-Pseudomonas aeruginosa vaccine, or the like.

### BACKGROUND ART

With highly advanced medical care and an ageing population, many fatal infections caused by multidrug-resistant bacteria have been reported. In particular, there has been rapid progress in the development of advanced multidrug resistance in Pseudomonas aeruginosa, which is a highly prevalent organism causing opportunistic infections and ventilator-associated pneumonia (target for intensive care), for which there is a strong need to develop vaccines that strengthen immunity without relying on antimicrobial drugs.

Patent Literature 1 discloses immunotherapy using PcrV, a constituent of the type III secretion system. Patent Literature 2 discloses that a fusion protein of OprF and OprI, which is an outer membrane component protein, is effective as an anti-Pseudomonas aeruginosa vaccine.

### CITATION LIST

### Patent Literature

PTL 1: WO2000/33872
PTL 2: WO2012/084272

### SUMMARY OF INVENTION

### Technical Problem

An object of the present invention is to provide a protein molecule effective for an anti-Pseudomonas aeruginosa vaccine.

### Solution to Problem

In the course of research, the present inventor came to focus on factors that seem to be unrelated to each other, namely PcrV, a component of the type III secretion system, OprF, an outer membrane component protein, and Exotoxin A, an exotoxin secreted by the type II secretion system. In view of the problem described above, the inventor conducted extensive research and found that excellent anti-Pseudomonas aeruginosa vaccine effects can be obtained by combining a PcrV antigen with at least one member selected from the group consisting of an OprF antigen and an Exotoxin A antigen. The inventor conducted further extensive research based on the above finding, and accomplished the present invention. Specifically, the present invention includes the following aspects.

Item 1. A protein molecule comprising PcrV antigen domain and at least one domain selected from the group consisting of OprF antigen domains and Exotoxin A antigen domains.
Item 2. The protein molecule according to Item 1, wherein the domains are linked individually via a linker.
Item 3. The protein molecule according to Item 1 or 2, comprising the PcrV antigen domain, OprF antigen domain, and Exotoxin A antigen domain.
Item 4. The protein molecule according to Item 3, wherein the PcrV antigen domain, OprF antigen domain, and Exotoxin A antigen domain are arranged in this order.
Item 5. The protein molecule according to any one of Items 1 to 4, which is a fusion protein.
Item 6. The protein molecule according to Item 5, wherein the number of amino acid residues is 700 or less.
Item 7. A polynucleotide comprising a coding sequence of the protein molecule according to Item 5 or 6.
Item 8. A composition comprising at least one member selected from the group consisting of a PcrV antigen and a polynucleotide comprising a PcrV antigen-coding sequence, and at least one member selected from the group consisting of an OprF antigen, a polynucleotide comprising an OprF antigen-coding sequence, an Exotoxin A antigen, and a polynucleotide comprising an Exotoxin A antigen-coding sequence.
Item 9. A drug comprising at least one member selected from the group consisting of the protein molecule according to any one of Items 1 to 6, the polynucleotide according to Item 7, and the composition according to Item 8.
Item 10. The drug according to Item 9, which is an anti-Pseudomonas aeruginosa vaccine.
Item 11. A drug comprising an antibody against a PcrV antigen, and at least one member selected from the group consisting of an antibody against an OprF antigen and an antibody against an Exotoxin A antigen.
Item 12. The drug according to Item 11, which is for use in the treatment of Pseudomonas aeruginosa infection.

### Advantageous Effects of Invention

The present invention provides a protein molecule, a polynucleotide, and a composition, each being effective for an anti-Pseudomonas aeruginosa vaccine, a combination of antibodies effective for the treatment of Pseudomonas aeruginosa infection, etc.

### BRIEF DESCRIPTION OF DRAWINGS

FIG. 1 shows a vector map of a vector for generation of a gene-recombinant three-antigen protein POmT.

FIG. 2 shows a CBB-stained image of SDS-PAGE gel of a sample in each stage in the purification of the gene-recombinant three-antigen protein POmT.

FIG. 3 shows the results of antibody titer measurement (Test Example 1). The horizontal axis shows samples administered as vaccines. The bars indicate antigen samples used for ELISA assay.

FIG. 4 shows the mouse survival rate after pulmonary administration of Pseudomonas aeruginosa. The horizontal axis shows the time elapsed from the administration of Pseudomonas aeruginosa. The lines indicate samples administered as vaccines.

FIG. 5 shows the quantification results of pulmonary edema in mice after pulmonary administration of Pseudomonas aeruginosa. The vertical axis shows pulmonary edema (the ratio of wet weight to dry weight) and the horizontal axis shows tested groups (demonstrating the sample name administered as a vaccine).

### DESCRIPTION OF EMBODIMENTS

### 1. Definition

In the present specification, the terms “comprising,” “containing,” and “including” include the concepts of comprising, containing, consisting essentially of, and consisting of.

In the present specification, the “identity” of amino acid sequences refers to the degree to which two or more contrastable amino acid sequences match each other. Thus, the higher the degree of match between two amino acid sequences, the higher the identity or similarity of those sequences. The level of amino acid sequence identity is determined, for example, by using FASTA (a tool for sequence analysis) with default parameters. Alternatively, the level of amino acid sequence identity can be determined by using the BLAST algorithm by Karlin and Altschul (Karlin S, Altschul SF. Methods for assessing the statistical significance of molecular sequence features by using general scorings schemes, Proc Natl Acad Sci USA. 87: 2264-2268(1990), Karlin S, Altschul SF. Applications and statistics for multiple high-scoring segments in molecular sequences, Proc Natl Acad Sci USA. 90: 5873-7(1993)). A program called “BLASTX,” based on this BLAST algorithm, has been developed. The specific techniques of these analysis methods are known and can be found on the website of the National Center of Biotechnology Information (NCBI) (http://www.ncbi.nlm.nih.gov/). The “identity” of base sequences is also defined in the same manner as above.

In the present specification, “conservative substitution” means the substitution of an amino acid residue with an amino acid residue having a similar side chain. For example, the substitution between amino acid residues having a basic side chain such as lysine, arginine, or histidine is considered to be a conservative substitution. The following substitutions between other amino acid residues are also considered to be a conservative substitution: the substitution between amino acid residues having an acidic side chain such as aspartic acid or glutamic acid; the substitution between amino acid residues having an uncharged polar side chain such as glycine, asparagine, glutamine, serine, threonine, tyrosine, or cysteine; the substitution between amino acid residues having a nonpolar side chain such as alanine, valine, leucine, isoleucine, proline, phenylalanine, methionine, or tryptophan; the substitution between amino acid residues having a β-branched side chain such as threonine, valine, or isoleucine; and the substitution between amino acid residues having an aromatic side chain such as tyrosine, phenylalanine, tryptophan, or histidine.

In the present specification, polynucleotides, such as DNA or RNA, may be chemically modified DNA or RNA as described below. To prevent the degradation by hydrolases such as nucleases, the phosphate residue (phosphate) of each nucleotide can be substituted with, for example, a chemically modified phosphate residue such as phosphorothioate (PS), methylphosphonate, or phosphorodithionate. The hydroxyl group at position 2 of the ribose of each ribonucleotide may also be substituted with —OR (R represents, for example, CH₃(2′-O-Me), CH₂CH₂OCH₃(2′-O-MOE), CH₂CH₂NHC(NH)NH₂, CH₂CONHCH₃, or CH₂CH₂CN). Additionally, the base moiety (pyrimidine, purine) may be chemically modified, by, for example, introduction of a methyl group or a cationic functional group into positon 5 of the pyrimidine base, or substitution of the carbonyl group at position 2 with thiocarbonyl. Additionally, the polynucleotide of the present invention also includes, but is not limited to, those formed by modifying the phosphate moiety or the hydroxyl portion, for example, by biotin, an amino group, a lower alkyl amine group, or an acetyl group. A BNA (LNA) and the like, in which the conformation of the sugar moiety is immobilized in the N form by bridging the 2′oxygen and 4′carbon in the sugar moiety of the nucleotide, can also be used.

In the present specification, the “coding sequence” is not particularly limited as long as it is a nucleotide sequence encoding an amino acid sequence of a protein.

### 2. Protein Molecule

In one embodiment, the present invention relates to a protein molecule (in this specification, sometimes referred to as “protein molecule of the invention”) comprising PcrV antigen domain and at least one domain selected from the group consisting of OprF antigen domains and Exotoxin A antigen domains. This is explained below.

The PcrV antigen is not particularly limited as long as it is a peptide comprising a full or partial amino acid sequence of PcrV protein. The PcrV antigen domain is a partial domain comprising the protein molecule of the present invention, and is a domain comprising a PcrV antigen.

The PcrV protein is a protein constituting the type III secretion system of Pseudomonas aeruginosa, and is one of the proteins that constitute the needle tip region of the type III secretion apparatus.

The Pseudomonas aeruginosa strain derived from the PcrV protein is not particularly limited. Examples of the Pseudomonas aeruginosa strain include PA103, PAO1, PA14, PACS2, PAK, C3719, VRFPA04, and the like.

The amino acid sequences of various PcrV proteins are known, or can be predicted from known amino acid sequences and based on genomic information of a strain (e.g., a known strain or a newly obtained strain), or can be identified by cloning based on known amino acid sequences. For example, the amino acid sequence of the PcrV protein in Pseudomonas aeruginosa strain PA103 is the amino acid sequence represented by SEQ ID No. 1, and the amino acid sequence of the PcrV protein in Pseudomonas aeruginosa strain PAO1 is the amino acid sequence represented by SEQ ID No. 14.

The partial amino acid sequence of the PcrV protein is not particularly limited as long as it is a contiguous amino acid sequence of sufficient length to be used as a PcrV antigen, i.e., to induce an antibody in the PcrV protein amino acid sequence. The number of constituent amino acid residues of the partial amino acid sequence is, for example, 12 or more. In view of the performance of the protein molecule of the present invention as an anti-Pseudomonas aeruginosa vaccine, the number of amino acid residues is preferably 20 or more, more preferably 50 or more, even more preferably 100 or more, still more preferably 150 or more, and further preferably 200 or more.

The partial amino acid sequence of the PcrV protein preferably includes the C-terminal region, e.g., the amino acid region 144^{th} to 257^{th} from the N-terminus of SEQ ID No. 1 or No. 14, in view of the performance of the protein molecule of the present invention, as an anti-Pseudomonas aeruginosa vaccine.

The full or partial amino acid sequence of the PcrV protein may have an amino acid substitution, deletion, addition, insertion, and like mutation against the full or partial amino acid sequence of the wild-type PcrV protein. The mutation is preferably a substitution, and more preferably a conservative substitution. If the full or partial amino acid sequence of the PcrV protein has an amino acid mutation, it has, for example, at least 90%, preferably at least 95%, more preferably at least 98%, and even more preferably at least 99% identity with the full or partial amino acid sequence of the wild PcrV protein.

Preferable examples of the full or partial amino acid sequence of PcrV protein include the amino acid sequences described in (a) and (b) below:

At least one member selected from the group consisting of

(a) the full or partial amino acid sequence of a wild PcrV protein (e.g., the amino acid sequence represented by SEQ ID No. 1 or No. 14, or a partial amino acid sequence thereof), and (b) the amino acid sequence having at least 90% identity with the full or partial amino acid sequence of a wild PcrV protein (e.g., the amino acid sequence represented by SEQ ID No. 1 or No. 14, or a partial amino acid sequence thereof).

In (b) above, the identity is preferably at least 95%, more preferably at least 98%, and even more preferably at least 99%. The number of amino acids mutated in (b) above is preferably 1 to 20, more preferably 1 to 10, even more preferably 1 to 5, and still more preferably 1 to 2.

The number of PcrV antigen domains contained in the protein molecule of the present invention is typically one, but can be two or more (e.g., 2 to 6, 2 to 4, or 2 to 3). However, from the viewpoint of suppressing the molecular weight of the protein molecule of the present invention and facilitating the preparation, administration, and metabolism after administration, the number of PcrV antigen domains is preferably small, and particularly preferably one.

The OprF antigen is not particularly limited as long as it is a peptide comprising the full or partial amino acid sequence of the OprF protein. The OprF antigen domain is a partial domain constituting the protein molecule of the present invention, and is a domain comprising an OprF antigen.

The OprF protein is a protein composing porin of the outer membrane of Pseudomonas aeruginosa.

The Pseudomonas aeruginosa strain from which the OprF protein is derived is the same as the Pseudomonas aeruginosa strain from which the PcrV protein is derived.

The amino acid sequences of various OprF proteins are known or can be predicted from known amino acid sequences and based on genomic information of a strain (e.g., a known strain or a newly obtained strain), or can be identified by cloning based on known amino acid sequences. For example, the amino acid sequence of the OprF protein in Pseudomonas aeruginosa strains PA103 and PA01 is the amino acid sequence represented by SEQ ID No. 15, and a mature amino acid sequence thereof (e.g., an amino acid sequence from which a signal peptide sequence is removed).

The partial amino acid sequence of the OprF protein is not particularly limited as long as it is a contiguous amino acid sequence of sufficient length to be used as an OprF antigen, i.e., to induce an antibody in the OprF protein amino acid sequence. The number of constituent amino acid residues of the partial amino acid sequence is, for example, 12 or more. In terms of the performance of the protein molecule of the present invention as an anti-Pseudomonas aeruginosa vaccine, the number of amino acid residues is preferably 20 or more, more preferably 50 or more, even more preferably 100 or more, and still more preferably 120 or more.

In terms of suppressing the molecular weight of the protein molecule of the present invention, and facilitating the preparation, administration, and metabolism after administration, the partial amino acid sequence of the OprF protein is preferably used as an OprF antigen, in which case, the number of amino acid residues is preferably 250 or less, more preferably 200 or less, even more preferably 170 or less, and still more preferably 160 or less.

The partial amino acid sequence of the OprF protein preferably includes a region containing an intermediate domain, e.g., the amino acid region 198^{th} to 342^{nd} (e.g., SEQ ID No. 3) from the N-terminus of SEQ ID No. 15, in view of the performance of the protein molecule of the present invention as an anti-Pseudomonas aeruginosa vaccine.

The full or partial amino acid sequence of the OprF protein may have an amino acid substitution, deletion, addition, insertion, and like mutation against the full or partial amino acid sequence of the wild-type OprF protein. The mutation is preferably a substitution, and more preferably a conservative substitution. If the full or partial amino acid sequence of the OprF protein has an amino acid mutation, it has, for example, at least 90%, preferably at least 95%, more preferably at least 98%, and even more preferably at least 99% identity with the full or partial amino acid sequence of the wild OprF protein.

Preferable examples of the full or partial amino acid sequence of OprF protein include the amino acid sequences described in (c) and (d) below:

At least one member selected from the group consisting of

(c) the full or partial amino acid sequence of a wild OprF protein (e.g., the amino acid sequence represented by SEQ ID No. 15, or a partial amino acid sequence thereof, or the amino acid sequence comprising the amino acid sequence represented by SEQ ID No. 3), and
(d) the amino acid sequence having at least 90% identity with the full or partial amino acid sequence of a wild OprF protein (e.g., the amino acid sequence represented by SEQ ID No. 15, or a partial amino acid sequence thereof, or the amino acid sequence comprising the amino acid sequence represented by SEQ ID No. 3).

In (d) above, the identity is preferably at least 95%, more preferably at least 98%, and even more preferably at least 99%. The number of amino acids mutated in (d) above is preferably 1 to 20, more preferably 1 to 10, even more preferably 1 to 5, and still more preferably 1 to 2.

The number of OprF antigen domains contained in the protein molecule of the present invention is typically one, but can be two or more (e.g., 2 to 6, 2 to 4, or 2 to 3). However, from the viewpoint of suppressing the molecular weight of the protein molecule of the present invention and facilitating the preparation, administration, and metabolism after administration, the number of OprF antigen domains is preferably small, and particularly preferably one.

The Exotoxin A antigen is not particularly limited as long as it is a peptide comprising the full or partial amino acid sequence of Exotoxin A protein. The Exotoxin A antigen domain is a partial domain constituting the protein molecule of the present invention, and a domain comprising the Exotoxin A antigen.

The Exotoxin A protein is the main cytotoxic in which Pseudomonas aeruginosa is secreted in a type II secretion mode.

The Pseudomonas aeruginosa strain from which the Exotoxin A protein is derived is the same as the Pseudomonas aeruginosa strain from which the PcrV protein is derived.

The amino acid sequences of various Exotoxin A proteins are known or can be predicted from known amino acid sequences and can be predicted based on genomic information of a strain (e.g., a known strain or a newly obtained strain), or can be identified by cloning based on known amino acid sequences. For example, the amino acid sequence of the Exotoxin A protein in Pseudomonas aeruginosa strain PA103 is the amino acid sequence represented by SEQ ID No. 16, and mature amino acid sequence thereof (e.g., an amino acid sequence from which a signal peptide sequence is removed).

The partial amino acid sequence of the Exotoxin A protein is not particularly limited as long as it is a contiguous amino acid sequence of sufficient length to be used as an Exotoxin A, i.e., to induce an antibody in the Exotoxin A protein amino acid sequence. The number of constituent amino acid residues of the partial amino acid sequence is, for example, 12 or more. In terms of the performance of the protein molecule of the present invention as an anti-Pseudomonas aeruginosa vaccine, the number of amino acid residues is preferably 20 or more, more preferably 50 or more, even more preferably 100 or more, still more preferably 150 or more, and further more preferably 200 or more.

From the viewpoint of suppressing the molecular weight of the protein molecule of the present invention and facilitating the preparation, administration, and metabolism after administration, the partial amino acid sequence of the Exotoxin A protein is preferably used as an Exotoxin A antigen. In this case, the number of amino acid residues is preferably 500 or less, more preferably 400 or less, even more preferably 300 or less, and further preferably 250 or less.

The partial amino acid sequence of the Exotoxin A protein preferably includes a region containing an enzyme active site, e.g., the amino acid region 413^{rd} to 638^{th} from the N-terminus of SEQ ID No. 16 (e.g., SEQ ID No. 5), in view of the performance of the protein molecule of the present invention, as an anti-Pseudomonas aeruginosa vaccine.

The full or partial amino acid sequence of the Exotoxin A protein may have an amino acid substitution, deletion, addition, insertion, and like mutation against the full or partial amino acid sequence of the wild-type Exotoxin A protein. The mutation is preferably a substitution, and more preferably a conservative substitution. If the full or partial amino acid sequence of the Exotoxin A protein has an amino acid mutation, it has, for example, at least 90%, preferably at least 95%, more preferably at least 98%, and even more preferably at least 99% identity with the full or partial amino acid sequence of the wild Exotoxin A protein.

In the full or partial amino acid sequence of the Exotoxin A protein, the enzyme-active amino acid of Exotoxin A (in the case of SEQ ID No. 16, the 578^{th} amino acid (glutamic acid) from the N-terminus) is preferably deleted. This can reduce side effects in the administration subject.

Preferable examples of the full or partial amino acid sequence of Exotoxin A protein include the amino acid sequences described in (e) and (f) below:

At least one member selected from the group consisting of

(e) the full or partial amino acid sequence of wild Exotoxin A protein (e.g., the amino acid sequence represented by SEQ ID No. 16, or a partial amino acid sequence thereof, or the amino acid sequence comprising the amino acid sequence represented by SEQ ID No. 5), and
(f) the amino acid sequence having at least 90% identity with the full or partial amino acid sequence of wild Exotoxin A protein (e.g., the amino acid sequence represented by SEQ ID No. 16, or a partial amino acid sequence thereof, or the amino acid sequence comprising the amino acid sequence represented by SEQ ID No. 5).

In (f) above, the identity is preferably at least 95%, more preferably at least 98%, and even more preferably at least 99%. The number of mutated amino acids in (d) above is preferably 1 to 20, more preferably 1 to 10, even more preferably 1 to 5, and still more preferably 1 to 2.

The number of Exotoxin A antigen domains contained in the protein molecule of the present invention is typically one, but can be two or more (e.g., 2 to 6, 2 to 4, and 2 to 3). However, from the viewpoint of suppressing the molecular weight of the protein molecule of the present invention, and facilitating preparation, administration, and metabolism after administration, the number of Exotoxin A antigen domains is preferably small, and particularly preferably one.

In the protein molecule of the present invention, the domains (e.g., between the PcrV antigen domain and the OprF antigen domain, between the PcrV antigen domain and the Exotoxin A antigen domain, between the OprF antigen domain and the Exotoxin A antigen domain, etc.,) are each directly or indirectly linked.

When the domains are directly linked, the linkage may be due to peptide bonding or other chemical cross-linking (e.g., carbodiimide chemistry, reductive animation, cyanylation chemistry (e.g., CDAP chemistry), maleimide chemistry, hydrazide chemistry, ester chemistry, and N-hydroxy succinic acid imide chemistry.)

When the domains are indirectly linked, they are preferably linked via a linker. Examples of the linker include peptide linkers, chemically cross-linked linkers (e.g., linkers having 4 to 12 carbon atoms, bifunctional linkers, linkers containing one or two reactive amino groups at the end, B-proprionamido, nitrophenylethylamine, haloacyl halide, 6-aminocaproic acid, and ADH). Preferable examples include peptide linkers.

In the protein molecule of the present invention, the domains are each preferably linked via a linker. In particular, it is particularly preferable in the protein molecule of the present invention that the domains are each linked via a peptide linker (i.e., the protein molecule of the present invention is a fusion protein). This allows the protein molecule of the present invention to be stably isolated and purified using genetic recombination technology, enabling achievement of high performance as an anti-Pseudomonas aeruginosa vaccine.

The peptide linker is not particularly limited as long as it functions as a linker. It is preferable that the peptide linker is an amino acid sequence that does not foam a secondary structure, such as α-helix structure or β-sheet structure, and that it is freely foldable. The peptide linker is preferably an amino acid sequence consisting of units of several amino acid residues linked. The peptide linker is preferably a linker (GS linker) comprising glycine and serine. Specific examples include GGGGS (SEQ ID No. 17), GSG, SAGG (SEQ ID No. 18), GGGS (SEQ ID No. 19), or GGSG (SEQ ID No. 20) as a constituent unit. The number of amino acid residues in the peptide linker is not particularly limited, and is, for example, 3 or more, preferably 3 to 20, and more preferably 4 to 10. By keeping the number of amino acid residues in the peptide linker at this level, the molecular weight of the protein molecule of the present invention can be reduced, and the preparation, administration, and metabolism after administration can be facilitated.

In view of the performance of the protein molecule of the present invention as an anti-Pseudomonas aeruginosa vaccine, it is particularly preferred to contain three domains (PcrV antigen domain, OprF antigen domain, and Exotoxin A antigen domain). In this case, from the same viewpoint, the PcrV antigen domain, the OprF antigen domain, and the Exotoxin A antigen domain are preferably arranged in this order. If the protein molecule of the present invention is a fusion protein, the PcrV antigen domain, the OprF antigen domain, and the Exotoxin A antigen domain are preferably arranged in this order from the N-terminus.

The protein molecule of the present invention may contain amino acid sequences other than those listed above (e.g., tag sequence and other antigen domain sequences).

The number of amino acid residues constituting the protein molecule of the present invention is preferably 1000 or less, more preferably 900 or less, even more preferably 800 or less, and particularly preferably 700 or less, from the viewpoint of facilitating preparation, administration, and metabolism after administration. In view of the performance as the anti-Pseudomonas aeruginosa vaccine, the number of amino acid residues is preferably 400 or more, more preferably 500 or more, and even more preferably 600 or more.

The protein molecule of the present invention may be chemically modified as long as the function as an anti-Pseudomonas aeruginosa vaccine is not greatly impaired.

The protein molecule of the present invention may have a carboxyl group (—COOH), carboxylate (—COO⁻), amide (—CONH₂), or ester (—COOR) at the C-terminus.

“R” in the ester is, for example, a C₁₋₆ alkyl group such as methyl, ethyl, n-propyl, isopropyl, or n-butyl; a C₃₋₈ cycloalkyl group such as cyclopentyl or cyclohexyl; a C₆₋₁₂ aryl group such as phenyl or α-naphthyl; a phenyl-C₁₋₂ alkyl group such as benzyl or phenethyl; a C₇₋₁₄ aralkyl group such as an α-naphthyl-C₁₋₂ alkyl group such as α-naphthyl methyl; or a pivaloyloxymethyl group.

The protein molecule of the present invention may have an amidated or esterified carboxyl group (or carboxylate), which is not the carboxyl group at the C-terminus. The ester in this case may be, for example, the esters of the C-terminus described above.

The protein molecule of the present invention further includes molecules having the amino group of the N-terminal amino acid residue protected by a protective group (e.g., a C₁₋₆ acyl group including a C₁₋₆ alkanoyl such as a formyl group and an acetyl group), molecules having the N-terminal glutamine residue pyroglutamated that can be formed due to cleavage in vivo; molecules having a substituent (e.g., —OH, —SH, an amino group, an imidazole group, an indole group, and a guanidino group) on a side change of an amino acid in the molecule protected by an appropriate protective group (e.g., a C₁₋₆ acyl group including a C₁₋₆ alkanoyl group such as a formyl group and an acetyl group); and the like.

The protein molecule of the present invention may be a pharmaceutically acceptable salt formed with an acid or base. The salt can be any pharmaceutically acceptable salt, and can be either an acid salt or a basic salt. Examples of acid salts include inorganic acid salts, such as hydrochloride, hydrobromide, sulfate, nitrate, and phosphate; organic acid salts, such as acetate, propionate, tartarate, fumarate, maleate, malate, citrate, methanesulfonate, and para-toluenesulfonate; and amino acid salts, such as aspartate and glutamate. Examples of basic salts include alkali metal salts such as sodium salts and potassium salts; and alkaline-earth metal salts such as calcium salts and magnesium salts.

The protein molecule of the present invention may be in the form of a solvate. The solvent can be any pharmaceutically acceptable solvent, and may be, for example, water, ethanol, glycerol, or acetic acid.

The protein molecule of the present invention can be easily prepared according to a known genetic engineering method depending on its amino acid sequence. For example, the protein molecule can be prepared using PCR, restriction enzyme cleavage, a DNA ligation technique, an in vitro transcription/translation technique, a recombinant protein production technique, etc.

The protein molecule of the present invention may be purified after synthesis. The cells may be recovered from the culture, for example, by centrifugation, filtration, etc., and extracted to give the protein molecule of the present invention. At the first step of extraction, the cells may be destroyed by digestion with an enzyme, destruction with osmotic pressure, sudden pressure and decompression, sonication, use of various homogenizers, etc. The destroyed cells are then fractionated by physical means such as low-speed centrifugation, ultra-centrifugation, filtration, a molecular sieve, membrane concentration, etc.; or by chemical means such as a precipitating agent, a solubilizing agent, an adsorbent and desorption agent, a dispersing agent, etc.; or by physicochemical means such as electrophoresis, column chromatography, a support, dialysis, salting-out, etc. These techniques may be used in combination. In applying these techniques, physicochemical conditions, such as temperature, pressure, pH, and ion strength, can be suitably set.

### 3. Polynucleotide

In one embodiment, the present invention relates to a polynucleotide comprising the coding sequence of the protein molecule of the present invention, which is a fusion protein (in this specification, sometimes referred to as “polynucleotide of the present invention”). The polynucleotide is described below.

The coding sequence of the protein molecule of the present invention is not particularly limited as long as the polynucleotide comprises a base sequence encoding the protein molecule of the present invention.

In one embodiment, the polynucleotide of the present invention comprises an expression cassette of the protein molecule of the present invention.

The expression cassette of the protein molecule of the present invention is not particularly limited as long as it is a DNA that allows expression of the protein molecule of the present invention in cells. Typical examples of the expression cassette of the protein molecule of the present invention include a promoter and DNA comprising the coding sequence of the protein molecule of the present invention placed under the control of the promoter.

Living organisms from which target cells are derived are not particularly limited. Examples include bacteria such as Enterobacteriaceae, fungi such as yeast, animals, and plants. Examples of animals include various mammals such as humans, monkeys, mice, rats, dogs, cats, rabbits, pigs, horses, cows, sheep, goats, and deer; non-mammalian vertebrates and invertebrates; etc. In a preferable embodiment of the present invention, examples of living organisms from which target cells are derived include mammals, E. coli, yeast, Bacillus subtilis, zebrafish, Japanese medaka, and insects. The type of cells is not particularly limited, and cells derived from various tissues or cells having various properties can be used. Examples include a blood cell, a hematopoietic stem cell/progenitor cell, a gamete (a sperm, an ovum), a fibroblast cell, an epithelial cell, a vascular endothelial cell, a nerve cell, a liver cell, a keratin-generating cell, a muscle cell, an epidermal cell, an endocrine cell, an ES cell, an iPS cell, a tissue stem cell, and a cancer cell.

The promoter contained in the expression cassette of the protein molecule of the present invention may be any promoter and can be appropriately selected according to the target cells. The promoter for use may be, for example, any pol II promoter. Examples of pol II promoters include, but are not limited to, a CMV promoter, EF1 promoter, SV40 promoter, MSCV promoter, hTERT promoter, β-actin promoter, CAG promoter, etc. In addition, examples of promoters include a tryptophan promoter, such as trc and tac, lac promoter, T7 promoter, T5 promoter, T3 promoter, SP6 promoter, arabinose-induced promoter, cold-shock promoter, and tetracycline-induced promoter.

The expression cassette of the protein molecule of the present invention may comprise other elements (e.g., multiple cloning sites (MCS), drug resistance genes, replication origins, enhancer sequences, repressor sequences, insulator sequences, reporter protein (e.g., fluorescent protein)-coding sequences, and drug resistance-gene-coding sequences) as necessary. MCS is not particularly limited as long as it contains multiple (e.g., 2 to 50, preferably 2 to 20, and more preferably 2 to 10) restriction enzyme sites. If the protein molecule of the present invention does not contain a functional domain, the MCS can be used for insertion of the coding sequence of any functional domain.

Examples of drug resistance genes include chloramphenicol resistance genes, tetracycline resistance genes, neomycin resistance genes, erythromycin resistance genes, spectinomycin resistance genes, kanamycin resistance genes, hygromycin resistance genes, puromycin resistance genes, and the like.

The reporter protein is not particularly limited as long as it is a light-emitting (color-developing) protein capable of reacting with a specific substrate to emit light (develop a color) or a fluorescent protein capable of emitting fluorescence by the action of excited light. Examples of the light-emitting (color-developing) protein include luciferase, β-galactosidase, chloramphenicol acetyltransferase, and β-glucuronidase. Examples of the fluorescent protein include GFP, Azami-Green, ZsGreen, GFP2, HyPer, Sirius, BFP, CFP, Turquoise, Cyan, TFP1, YFP, Venus, ZsYellow, Banana, KusabiraOrange, RFP, DsRed, AsRed, Strawberry, Jred, KillerRed, Cherry, HcRed, and mPlum.

The expression cassette of the protein molecule of the present invention may constitute a vector by itself or in conjunction with another sequence. The type of vector is not particularly limited, and examples include plasmid vectors such as animal cell expression plasmids; virus vectors such as retroviruses, lentiviruses, adenoviruses, adeno-associated viruses, herpes viruses, and Sendai viruses; and the like. In addition to the above, examples of vectors include ColE1-based plasmid represented by pBR322 derivative, pACYC plasmid with p15A origin, pSC plasmid, and F factor-derived mini F plasmid such as Bac.

The polynucleotide of the present invention can be easily prepared according to a known genetic engineering method. For example, the polynucleotide can be prepared using PCR, restriction enzyme cleavage, a DNA ligation technique, etc.

### 4. Composition

In one embodiment, the present invention relates to a composition (in the specification, sometimes referred to as the “composition of the present invention”) comprising at least one member selected from the group consisting of a PcrV antigen and a polynucleotide comprising a PcrV antigen-coding sequence, and at least one member selected from the group consisting of an OprF antigen and a polynucleotide comprising an OprF antigen-coding sequence, an Exotoxin A antigen, and a polynucleotide comprising an Exotoxin A antigen-coding sequence.

Antigens and polynucleotides are the same as those described in the “2. Protein molecule” section and “Polynucleotide” section.

In view of the performance as the anti-Pseudomonas aeruginosa vaccine, the composition of the present invention preferably comprises at least one member selected from the group consisting of a PcrV antigen and a polynucleotide comprising a PcrV antigen-coding sequence, at least one member selected from the group consisting of an OprF antigen and a polynucleotide comprising an OprF antigen-coding sequence, and at least one member selected from the group consisting of an Exotoxin A antigen and a polynucleotide comprising an Exotoxin A antigen-coding sequence.

When two or more polynucleotides (e.g., the polynucleotide containing the PcrV antigen coding sequence and the polynucleotide containing the OprF antigen coding sequence) are contained, the polynucleotides can be linked to form a single polynucleotide molecule.

### 5. Drug 1

In one embodiment, the present invention relates to a drug (in the specification, sometimes referred to as “Drug 1 of the present invention”) comprising at least one member selected from the group consisting of the protein molecule of the present invention, the polynucleotide of the present invention, and the composition of the present invention (in this specification, sometimes referred to as the “active ingredients of the present invention.”) Drug 1 is explained below.

Drug 1 of the present invention is effective as a vaccine, particularly, an anti-Pseudomonas aeruginosa vaccine. Drug 1 of the present invention preferably contains the protein molecule of the present invention in view of the performance as an anti-Pseudomonas aeruginosa vaccine. Drug 1 of the present invention can prevent the development of Pseudomonas aeruginosa infection, e.g., sepsis, respiratory tract infection, urinary tract infection, bedsores, liver and biliary system infection, and gastrointestinal tract infection, and reduce the probability of death due to Pseudomonas aeruginosa infection.

The target organism of Drug 1 of the present invention is not particularly limited as long as it is a living organism that can be infected with Pseudomonas aeruginosa. Examples of the organism include various mammals, such as humans, monkeys, mice, rats, dogs, cats, and rabbits. Of these, humans are preferred because Drug 1 of the present invention efficiently exhibits its effects.

Drug 1 of the present invention is not particularly limited and may contain other components as necessary as long as it contains the active ingredient of the present invention. Examples of other components include a stabilizer for increasing the heat resistance of the vaccine and an adjuvant as an auxiliary for enhancing antigenicity.

As a stabilizer, for example, sugars or amino acids may be used. As an adjuvant, for example, aluminum compounds (e.g., aluminum hydroxide gel), CpG oligodeoxynucleotide, mineral oil, vegetable oil, alum, bentonite, silica, muramyl dipeptide derivatives, thymosin, interleukin, etc. may be used. Preferable examples of adjuvants include aluminum hydroxide gel, CpG oligodeoxynucleotide (e.g., K3 (K-type CpG ODN) and D35 (D-type CpG ODN), and the like. The adjuvants may be used alone or in a combination of two or more.

In addition to the above, examples of Drug 1 of the present invention include bases, carriers, solvents, dispersants, emulsifiers, buffers, stabilizers, excipients, binders, disintegrates, lubricants, thickeners, humectants, colorants, fragrances, chelating agents, and the like.

The dosage form of Drug 1 of the present invention is not particularly limited. Examples include an injection, including an aqueous injection, a non-aqueous injection, a suspension injection, and a solid injection; an oral preparation such as a tablet, a capsule, a granule, a powder, a fine granule, a syrup, an enteric preparation, a sustained-release capsule, a chewable tablet, drops, a pill, a liquid or solution for oral application, a lozenge, a sustained-release preparation, and a sustained-release granule; and a preparation for cutaneous application such as a nasal preparation, inhalation, a suppository for rectal application, a pessary, an enema, and a jelly.

The amount of the active ingredient of the present invention in Drug 1 of the present invention depends on the subject of administration, the route of administration, the dosage fault, the condition of the patient, and the determination of the doctor, and is not limited. For example, the amount of the active ingredient is, for example, 0.0001 to 95 wt %, and preferably 0.001 to 50 wt %.

The amount of Drug 1 of the present invention can be determined by a clinical physician, taking into consideration various factors, such as administration route, the health condition of the subject, the subject's age, sex, and body weight, pharmacological findings such as pharmacokinetics and toxicological characteristics, use or non-use of a drug delivery system, and whether Drug 1 is administered as part of a combinational drug with other medicinal agents. Drug 1 of the present invention is not particularly limited, but it is preferred that the dosage of the active ingredient of the present invention in a single administration, for example, is 1 μg to 10 mg/kg (body weight), and 50 to 1000 μg/kg (body weight). The interval and frequency of administration is not particularly limited, and the administration is preferably performed at an interval of about 1 week to 8 weeks for about 1 to 5 times.

### 6. Drug 2

In one embodiment, the present invention relates to a drug (in the specification, referred to as “Drug 2 of the present invention”) comprising an antibody against the PcrV antigen and at least one member selected from the group consisting of an antibody against the OprF antigen and an antibody against the Exotoxin A antigen (in the specification, referred to as the “antibody of the present invention.”) Drug 2 is explained below.

The antigens are the same as in the explanation in the “2. Protein molecule” section. Drug 2 of the present invention is effective as the drug for the treatment of Pseudomonas aeruginosa infection. From the viewpoint of treatment effects, Drug 2 of the present invention preferably contains the antibody against the PcrV antigen, antibody against the OprF antigen, and antibody against the Exotoxin A antigen. Drug 2 of the present invention enables the treatment (e.g., reduction, alleviation, or remission of symptoms) of Pseudomonas aeruginosa infection, such as sepsis, respiratory tract infection, urinary tract infection, bedsores, liver and biliary system infection, gastrointestinal tract infection, and the like.

Examples of the type of the antibody include a polyclonal antibody, a monoclonal antibody, a human chimeric antibody, a humanized antibody, a fully human antibody, a single-stranded antibody, or an antigen-binding fragment (Fab, F(ab′)2, minibody, scFv-Fc, Fv, scFv, diabody, triabody, and tetrabody.) The human chimeric antibody is an antibody in which the variable region of the antibody is a sequence derived from an animal other than a human (e.g. a mouse or cow), and the constant region of the antibody is a sequence derived from a human.

Methods for producing these antibodies are already well known, and the antibody of the present invention can also be produced in accordance with these ordinary methods (Current Protocols in Molecular Biology, Chapter 11.12 to 11.13 (2000)). Specifically, when the antibody of the present invention is a polyclonal antibody, the antigen can be immunized in a non-human animal such as a domestic rabbit, and the antibody can be obtained from the serum of the immunized animal according to a usual method. A monoclonal antibody as the antibody of the present invention can be obtained from hybridoma cells prepared by immunizing a non-human animal, such as a mouse or a cow, with the antigen (Current Protocols in Molecular Biology, edit. Ausubel et al. (1987), publish. John Wiley and Sons, Section 11.4 to 11.11).

The antibody can also be produced by increasing immunological reaction by using various adjuvants according to the host. Examples of such adjuvants include, but are not limited to, mineral gels such as Freund's adjuvant and aluminum hydroxide; surface-active substances such as lysolecithin, Pluronic Polyol, polyanion, peptide, oil emulsion, keyhole limpet hemocyanin, and dinitrophenol; and human adjuvants such as BCG (Bacille de Calmette et Guérin) and Corynebacterium parvum.

The additive, dosage form, and content of an active ingredient (antibody of the present invention) in Drug 2 of the present invention are the same as those in Drug 1 of the present invention.

The amount of Drug 2 in the present invention can be determined according to the type of usage. If Drug 2 is used for drugs, the amount can be determined by a clinical physician, taking into consideration various factors, such as the administration route, the type of disease, the degree of symptoms, patient's age, sex, and body weight, severity of disease, pharmacological findings such as pharmacokinetics and toxicological characteristics, use or non-use of a drug delivery system, and whether Drug 2 is administered as part of a combinational drug with other medicinal agents. Drug 2 of the present invention is not particularly limited. It is preferably used so that the administration amount of the antibody of the present invention per administration is 1 μg to 10 mg/kg (body weight). The interval and frequency of administration is not particularly limited; however, the administration can be performed once to several times (e.g., twice to ten times) per day or per month.

### EXAMPLES

The present invention is described in detail below with reference to Examples. However, the present invention is not limited to these Examples.

### Example 1. Preparation of Three Antigen Complex Protein Molecules

PcrV, OprF, and Exotoxin A were selected from antigen proteins involving Pseudomonas aeruginosa virulence. As an OprF antigen, the partial polypeptide (OprF₁₉₈₋₃₄₂) of OprF protein was used. As an Exotoxin A antigen, a partial polypeptide (mTox_{413-Δ578E-638}) obtained by removing #578 glutamic acid, which was an enzyme active amino acid, by a genetic modification technique from the ToxA₄₁₃₋₅₃₈ domain of the C-terminal region containing the enzyme active site of the Exotoxin A protein.

The total of three gene regions, comprising pcrV₁₋₈₈₂ (nucleotide sequence: SEQ ID No. 2) encoding the full-length of PcrV₁₋₂₉₄ (amino acid sequence: SEQ ID No. 1), oprF₅₉₄₋₁₀₂₈ (nucleotide sequence: SEQ ID No. 4) encoding the intermediate domain region of OprF, OprF₁₉₈₋₃₄₂ (amino acid sequence: SEQ ID No.3), and mTox₁₂₃₉₋₁₉₁₆ (nucleotide sequence: SEQ ID No. 6) encoding the C-terminal domain of Exotoxin A, ToxA₄₁₃₋₆₃₈ (amino acid sequence: SEQ ID No. 5) was cloned from P. aeruginosa strain PA103 chromosomal DNA. For ToxA₄₁₃₋₆₃₈, #578 glutamic acid was removed by a genetic modification technique (amino acid sequence: SEQ ID No. 7, nucleotide sequence: SEQ ID No. 8).

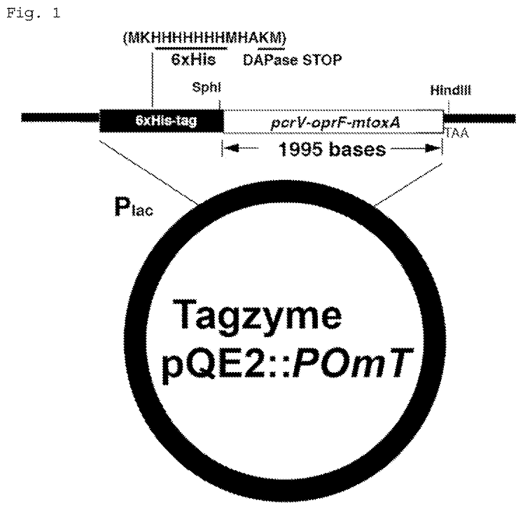

The three resulting gene regions were linked using the polymerase chain reaction technique by using linker Oligo DNA encoding glycine serine linkers (-GGGGS- (SEQ ID No.9) and -GSGGSG- (SEQ ID No.10)). DNA (nucleotide sequence: SEQ ID No.12) encoding a protein (amino acid sequence: SEQ ID No. 11) (abbreviated as “POmT”) comprising PcrV₁₋₂₉₄, a GGGGS linker, OprF₁₉₈₋₃₄₂, GSGGSG, and mTox_{413-Δ578E-638} in this order from the N-terminal side was prepared, and incorporated into a commercially available vector creation (TAGzyme (registered trademark) pQE2, QiAGen) for expression of a genetically modified E. coli protein, thus preparing a vector (TAGzyme pQE2::PomT) for generating a genetically modified three-antigen protein POmT without containing a purification tag (FIG. 1). The N-terminal 6×His tag amino acid sequence for purification of protein in the vector is “MKHHHHHHHHMHHAKM (SEQ ID No.13),” and the tag portion can be decomposed by DAPase enzyme treatment after purification in an IMAC column.

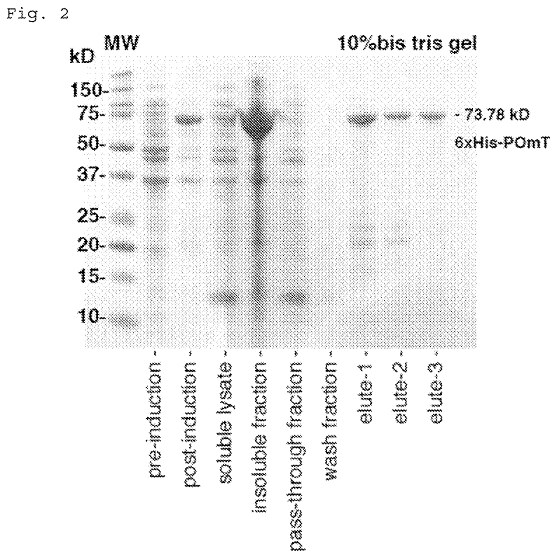

The vector was introduced into E. coli, and the resulting E. coli was cultured at 30° C. for 10 hours. The E. coli was then sonicated, and the crushed material was purified in the IMAC column to obtain 6×His-tagged POmT (FIG. 2). This was subjected to a DAPase enzyme treatment to decompose the tag portion, thus purifying a gene-recombinant three-antigen protein POmT.

### Comparative Example 1. Preparation of PcrV Antigen

A PcrV antigen was prepared by the same procedure as in Example 1, except that only the full-length of PcrV₁₋₂₉₄ cloned from Pseudomonas aeruginosa strain PA103 chromosomal DNA was used as an antigen coding sequence.

### Comparative Example 2. Preparation of OprF Antigen

An OprF antigen was prepared by the same procedure as in Example 1, except that only OprF₁₉₈₋₃₄₂ cloned from Pseudomonas aeruginosa strain PA103 chromosomal DNA was used as an antigen coding sequence.

### Comparative Example 3. Preparation of mTox Antigen

An mTox antigen was prepared by the same procedure as in Example 1, except that only mTox_{413-78E-638} cloned from Pseudomonas aeruginosa strain PA103 chromosomal DNA was used as an antigen coding sequence.

### Test Example 1. Evaluation of Vaccine Effect

The vaccine effect of the three-antigen protein POmT was evaluated using a mouse Pseudomonas aeruginosa pneumonia model. Specifically, the evaluation was performed as follows.

The purified vaccine protein (PomT, PcrV, OprF, or mTox; 10 μg/mouse) was absorbed into Alhydrogel (aluminum hydroxide gel adjuvant), and a protein-adjuvant mixture (100 uL) was injected intramuscularly into the thigh muscle of each mouse (POmT group: n=21, PcrV group: n=11). In the control group (n=18), an adjuvant alone was injected. Four weeks later, injection was performed again. Another four weeks later, antibody titer measurement and an infection experiment were performed.

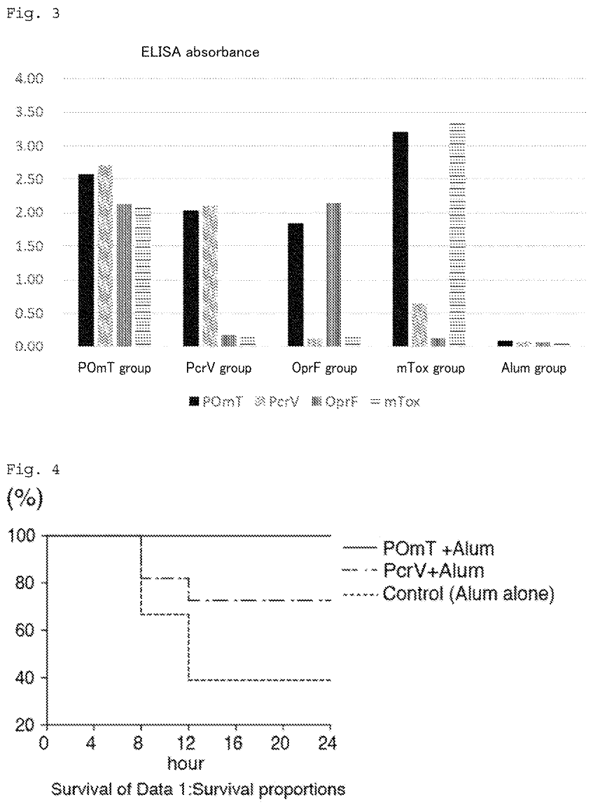

The antibody titer measurement was performed as follows. According to enzyme-linked immunosorbent assay (ELISA), the POmT protein, PcrV₁₋₂₉₄, OprF₁₉₈₋₃₄₂, and mTox_{413-Δ578E-638} were used as antigen proteins, and the titers of antibodies binding to these antigens were quantified by the colorimetric method. The results confirmed that the anti-PcrV titer, anti-OprF titer, and anti-mTox titer were all increased in mice immunized with the three-antigen vaccine protein POmT+Alum (FIG. 3).

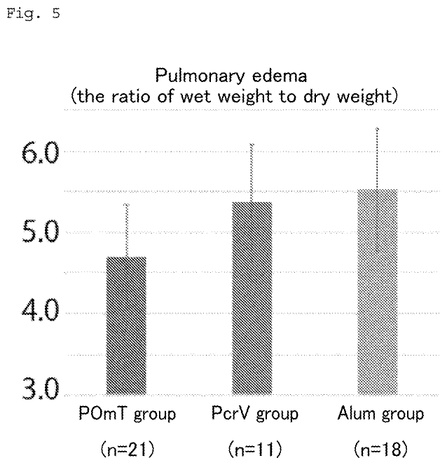

In the infection experiment, mice were subjected to general anaesthesia with sevoflurane inhalation for a short time period (about 1 minute), and 1×10⁶ Pseudomonas aeruginosa PA103 strains were intratracheally administered using a blunt needle. The mice were awakened after one minute and allowed to move freely in the cage. The number of survivors after 0 hours (before bacterial administration), 4 hours, 8 hours, 12 hours, and 24 hours, and the rectal temperature of the surviving mice was measured and counted as a 24-hour survival rate. The lungs of the mice were also removed after 24 hours and quantitatively homogenized to quantify edema. As a result, in mice to which a gene-recombinant antigen protein POmT containing three protein antigens had been administered, the survival rate 24 hours after the pulmonary administration of a lethal dose of Pseudomonas aeruginosa (PA103 strain, 1×10⁶ CFU/mouse) was significantly improved (p<0.0001) as compared to the PcrV single-administration group and the control group of the single administration with the Alhydrogen adjuvant (FIG. 4). Pulmonary edema was also significantly improved in the POmT administration group as compared to the two other groups (FIG. 5).

Sequence Listing

1. A protein molecule comprising PcrV antigen domain and at least one domain selected from the group consisting of OprF antigen domains and Exotoxin A antigen domains.

2. The protein molecule according to claim 1, wherein the domains are linked individually via a linker.

3. The protein molecule according to claim 1, comprising the PcrV antigen domain, OprF antigen domain, and Exotoxin A antigen domain.

4. The protein molecule according to claim 3, wherein the PcrV antigen domain, OprF antigen domain, and Exotoxin A antigen domain are arranged in this order.

5. The protein molecule according to claim 1, which is a fusion protein.

6. The protein molecule according to claim 5, wherein the number of amino acid residues is 700 or less.

7. A polynucleotide comprising a coding sequence of the protein molecule according to claim 5.

8. A composition comprising at least one member selected from the group consisting of a PcrV antigen and a polynucleotide comprising a PcrV antigen-coding sequence, and at least one member selected from the group consisting of an OprF antigen, a polynucleotide comprising an OprF antigen-coding sequence, an Exotoxin A antigen, and a polynucleotide comprising an Exotoxin A antigen-coding sequence.

9. A drug comprising the protein molecule according to claim 1.

10. The drug according to claim 9, which is an anti-Pseudomonas aeruginosa vaccine.

11. A drug comprising an antibody against a PcrV antigen, and at least one member selected from the group consisting of an antibody against an OprF antigen and an antibody against an Exotoxin A antigen.

12. The drug according to claim 11, which is for use in the treatment of Pseudomonas aeruginosa infection.
